(19) United States
(12) Patent Application Publication
(10) Pub. No.: US 2014/0100469 A1
(43) Pub. Date: Apr. 10, 2014

(54) INFLATABLE REMOTE SENSOR VEST SYSTEM FOR PATIENT MONITORING AND DIAGNOSIS

(71) Applicant: Boris Sagalovich, Brooklyn, NY (US)

(72) Inventor: Boris Sagalovich, Brooklyn, NY (US)

(21) Appl. No.: 13/645,885

(22) Filed: Oct. 5, 2012

(51) Int. Cl. A61B 5/00; A61B 7/04; A61B 5/02

(52) U.S. Cl. CPC A61B 5/6805; A61B 5/02; A61B 7/04
USPC 600/528; 600/300

(57) ABSTRACT

An inflatable vest system includes a wearable vest having an outer surface and an inner surface and a plurality of bladders having a hollow chamber. The bladders are dispersed at selected locations along the vest and disposed adjacent to each other, each having an inlet for selectively receiving and releasing air within its chamber. A plurality of adjustable sensors are attached to the vest and disposed within the vicinity of desired locations on the patient's body are provided for exerting pressure on the desired locations as a function of the air pressure of at least one bladder most closely located to the sensor. The vest receives control signals from a remote physician system that selectively activating and deactivating one of the sensors, and/or increase and decrease air pressures in desired one of the bladders.

### FIELD OF THE INVENTION

This invention relates to an inflatable vest for auscultation and other measurements via a remote control system.

### BACKGROUND

With the improved quality and speed and decreased cost of telecommunication systems, telemedicine has become a viable compliment and sometimes even a substitute to in-person health monitoring. Recently many experts in the medical field believe that a more routine and frequent monitoring and examination arrangement can predict early signs of an impending decline in a patient's health compared to a periodic check-up at specified intervals. Such routine and frequent monitoring may substantially prevent an onset of more serious health issues. However, the problem of frequent monitoring is inconvenience to the patient and added cost to the health care system.

For almost 200 years stethoscopes have become an integral tool for physicians diagnosing their patients. Over the years the design of stethoscopes improved by the introduction of the double sided chest-piece. The larger side was designed to allow the physician to listen to higher pitched sounds, and the small side was designed to filter out the higher pitched sounds. Single sided stethoscope is also frequently use with the application of the different pressure for lung and heard examination. Recently, with the advent of more powerful computing power, the sounds received by a stethoscope can be converted to electronic signals for storage, replay, and further software analysis.

Property performed, the auscultation examination is an inexpensive, widely available tool in the detection and management of heart and lung disease. Unfortunately, accurate interpretation of heart sounds by primary care providers is fraught with error, leading to missed diagnosis of disease and/or excessive costs associated with evaluation of normal variants. Furthermore, a patient may not exhibit symptoms at the time of a regularly scheduled examination at the doctor's office, and gradually develop symptoms that may be too late for taking precautionary measures by the time a follow up visit is scheduled.

Unfortunately even in telemedicine settings, in remote auscultation human assistance of the trained personal (nurse, medical assistant) is required in order to place stethoscope in appropriate parts of the body which add additional cost to telemedicine exam.

As such there is a need for a telemedicine system that allows a physician to perform cardiac and pulmonary auscultation on a patient remotely and more frequently so as to prevent impending medical conditions leading to better quality of life and less expensive treatments.

### SUMMARY

In accordance with one embodiment of the invention, a wearable bladder vest is provided to a patient that includes a plurality of air bladders, each of which may be controllable to inflate or deflate individually. The bladders have an inner side facing the patient's body and an outer side that faces outside. Individual sensors are disposed on predetermined locations on the inner side of the bladder vest. In one embodiment, the individual sensors may include those for performing auscultation. Each of the sensors and bladders are remotely activated or deactivated to allow a physician to remotely receive signals from sensors located within a desired location on the patient's body. The air pressure in the bladders can be varied to exert more or less pressure on the sensors located within the desired location on the patient's body. A controller system is attached to the bladder vest for providing control signals to individual sensors and for receiving electrical signals from the sensors. The controller also provides control signals for adjusting the air pressure in each of the individual bladders. The size of air bladders includes a wide range, starting as small as the size of each sensor, or large enough to cover many sensors in any given region corresponding to a region of a patient's body.

In another embodiment of the invention, an array of sensors arranged in a plurality of rows and columns are attached together to form a sensor vest. The sensor vest is configured to include sensors on both the front and the back side. The sensor vest is first worn by the patient, and adjusted for the accurate placement on the patient's body. Thereafter, the bladder vest is worn over the sensor vest, so as to provide localized pressure on the sensors of the sensor vest by selectively applying air pressure to desired locations on the bladder vest. In accordance with yet another embodiment of the invention, each sensor location includes an auscultation sensor or an EKG sensor or both. The sensor vest also includes a plurality of markers for aligning the sensor in a correct position when worn by a patient.

In accordance with another embodiment of the invention, a display system provides visual clues for the physician to allow the physician better control the sensors located at various locations with respect to the patient's body. In accordance with another embodiment, a touch screen arrangement allows the physician to visualize the patient's body with the worn vest. The physician is then enabled to press on the various locations on the image corresponding to the sensors located on the corresponding positions on the patient's body. In accordance with another embodiment, the physician may change the pressure on the screen by pressing his fingers harder or softer on the screen so as to visually control the amount of pressure is being exerted by the bladders located in the vicinity of the pressure sensed on the display screen.

During the operation, a patient visits a physician's office, or alternatively, a physician or an assistant visit the patient to provide a vest for the patient with the best fit. Thereafter, the location of the sensors are adjusted on the inside portion of the vest for the best location for their intended use. The patient is then trained to wear the vest for its optimum fit.

In accordance with another embodiment of the invention, a visual communication can also be established between the patient and the remote physician. A camera in front of the patient is enabled to transmit a video of the patient to the physician's display monitor and an audio visual communication is established between the two. As such, the physician may also instruct the patient on better adjusting the vest, if the signals received from the patient's sensor are not optimum because of the sub-optimum location of the sensors, after the vest is worn.

### BRIEF DESCRIPTION OF THE DRAWINGS

The present invention can be best understood through the following description and accompanying drawings, wherein:

FIG. 1a illustrates a front side of an inflatable bladder vest in accordance with one embodiment;

FIG. 1b illustrates a back side of an inflatable bladder vest in accordance with one embodiment;

FIG. 1c illustrates an additional front side of an inflatable bladder vest in accordance with one embodiment;

FIG. 1d illustrates a front side of an alternative vest with sensors physically attached together to form a sensor vest in accordance with one embodiment;

FIG. 1e illustrates a back side of an alternative vest with sensors physically attached together to form a sensor vest in accordance with one embodiment;

FIG. 1f illustrates a front/back side of an alternative vest with sensors physically attached together to form a sensor vest in accordance with one embodiment;

FIG. 2 is a close up view of a portion of a liner and bladders of the vest of FIG. 1, in accordance with one embodiment;

FIG. 3 shows an additional view of the location of sensors on the vest of FIG. 1, in accordance with one embodiment;

FIG. 4 illustrates a close up view of a stethoscope sensor, in accordance with one embodiment;

FIG. 5 is a block diagram of a controller of the vest of FIG. 1, in accordance with one embodiment;

FIG. 6 shows an exemplary physician station, in accordance with one embodiment; and

FIGS. 7a and 7b are a flow chart of the operation of the system, in accordance with one embodiment.

### DETAILED DESCRIPTION OF THE PREFERRED EMBODIMENT

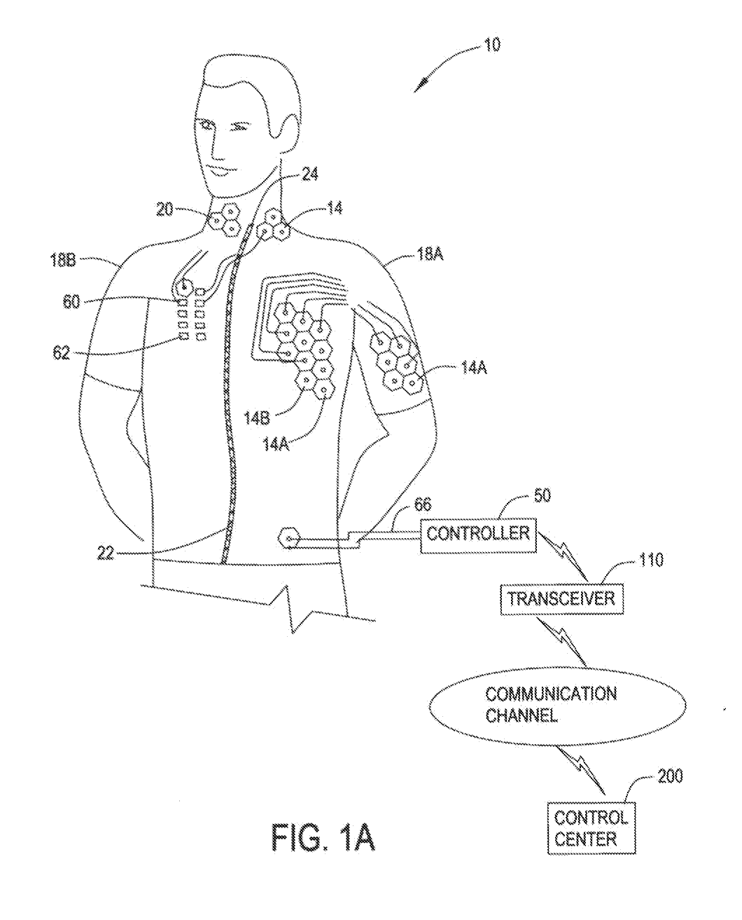

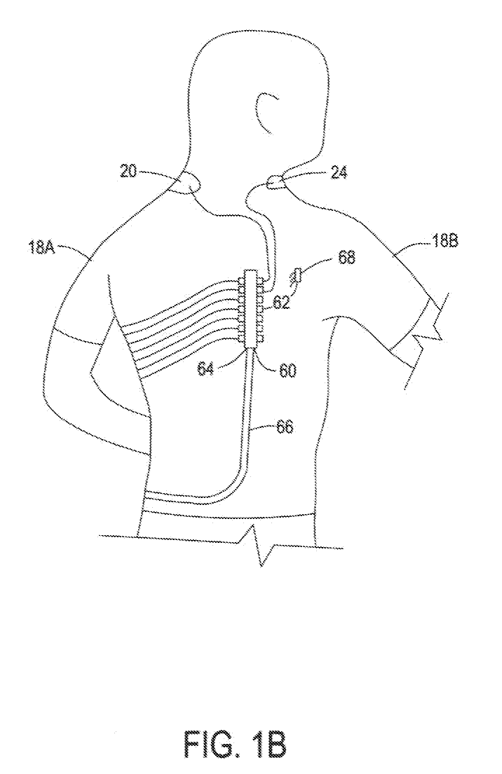

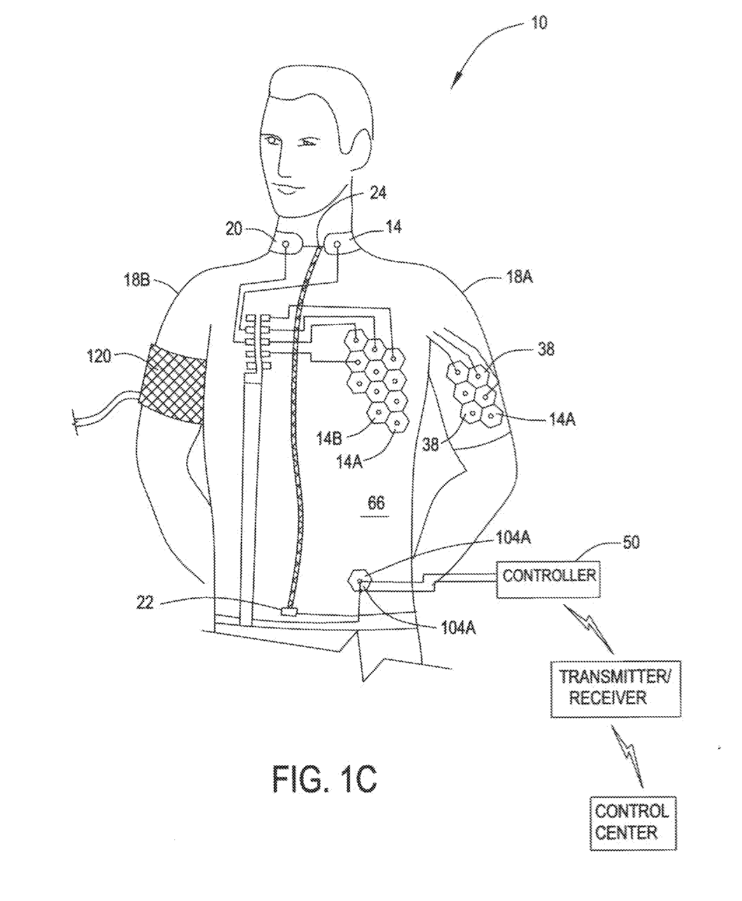

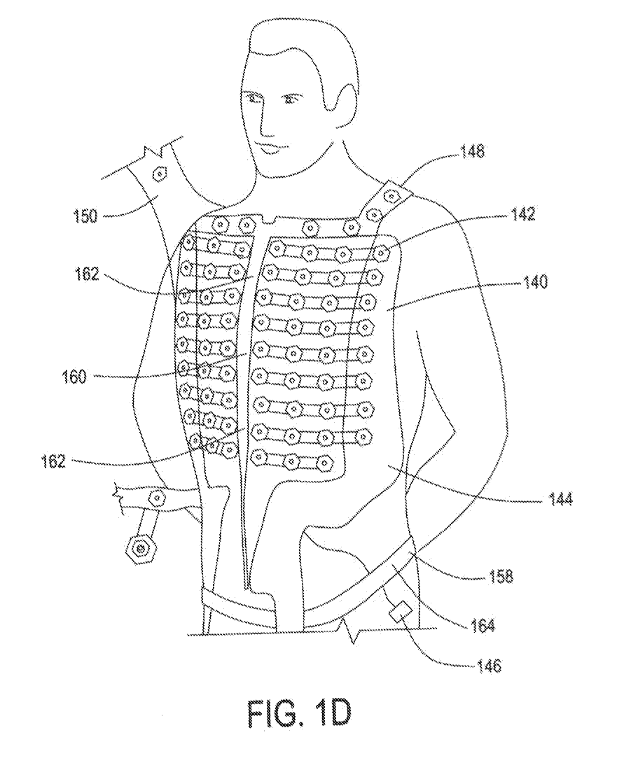

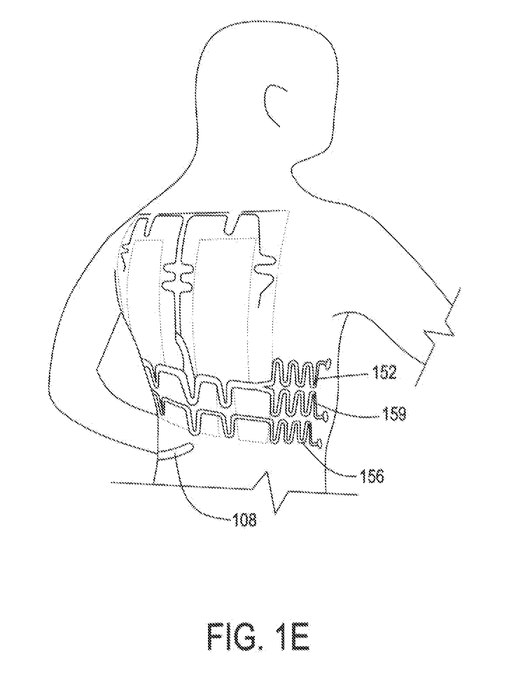

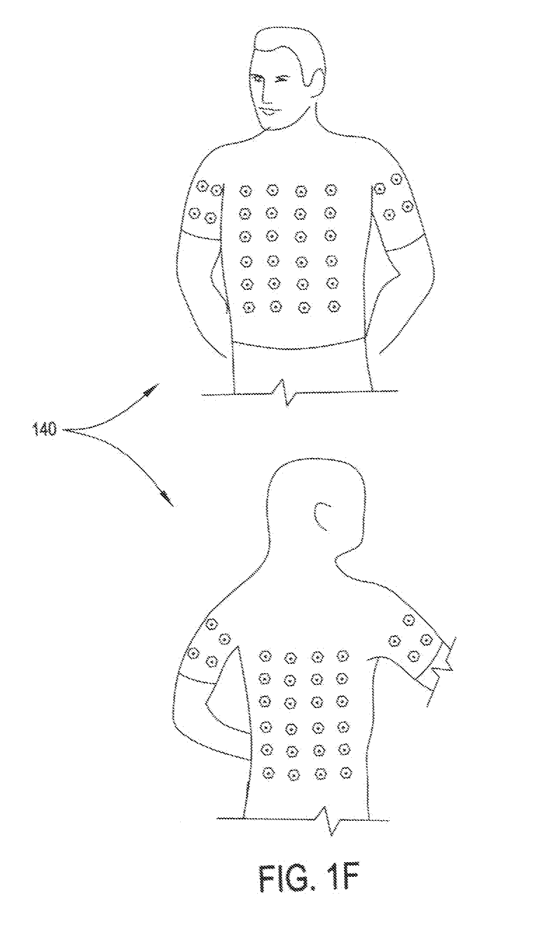

In accordance with one embodiment of the invention, FIGS. 1a and 1b, respectively illustrate the front and back side of an inflatable bladder vest 10 that can be worn by a patient 12. Depending on its intended use, vest 10 has a plurality of bladders 14a, 14b, . . . 14(n) all embedded within a liner 16 of vest 10.

Vest 10 includes arm sleeves 18a and 18 b, which extend at least below the elbow of patient 12. Vest 10 also includes a neck sleeve 20, within which additional bladders 14 are embedded. A zipper 22 allows the vest to close once it is worn by the patient. In accordance with one embodiment of the invention, zipper 22 can be substituted by other fastening arrangements, such as Velcro fasteners, or releasable buckles. The fastening arrangement may extend from the waist portion 22 of patient 12 to neck portion 24 of the patient. It is understood that vest 10 may be made of a solidly cap ed material or it may be partially and/or totally transparent.

It is noted that although the cellular bladder structure of one embodiment of the invention as depicted in FIG. 1 includes bladders 14 around the heart, left elbow and the neck portions of the patient, the invention is not limited in scope to such an arrangement. For example, in accordance with other embodiments of the invention, the cellular structure covers the back parts of the patient in addition to abdominal and kidney areas, right arm and other portions of the body, depending on the monitoring purpose of the vest. Furthermore, although the present embodiment employs cellular shaped bladders, the invention is not limited in scope in that respect, and other bladder shapes may be employed with various embodiments of the invention. The sizes of the bladders are varied also. Again, depending on the application, the size of the bladders may be comparable to the size of each individual sensor, or large enough to encompass many sensors at the same time with individual or group control as described in more detail below.

In accordance with one embodiment of the invention, and specifically for a bladder vest designed for auscultation, vest 10, in further reference with FIG. 3, includes bladders that substantially cover apex 26, which more accurately, covers the fifth intercostal space in the mid-clavicular line of the patient. This area is optimum for hearing mitral and left heart sounds. Vest 10 includes bladders around the left lateral sternal border 28 of the patient, which more accurately covers the fourth intercostal space to the left of the sternum of the patient. This area is optimum for hearing tricuspid and right heart sounds. Vest 10 also includes bladders around the base left 30 of the patient, which more accurately covers the second intercostal space to the left of the sternum. This area is optimum for hearing sounds from the pulmonic valve of the patient. Vest 10 further includes bladders around base right 32 of the patient, which more accurately covers the second intercostal space to the right of the sternum. This area is optimum for hearing sound from the aortic valve of the patient.

As will be explained in more detail, air bladders 14 can be individually controlled so as to allow auscultation of the patient in the supine, sitting and left lateral recumbent positions. In each of these positions, various sounds, especially abnormal sounds may be elicited more clearly.

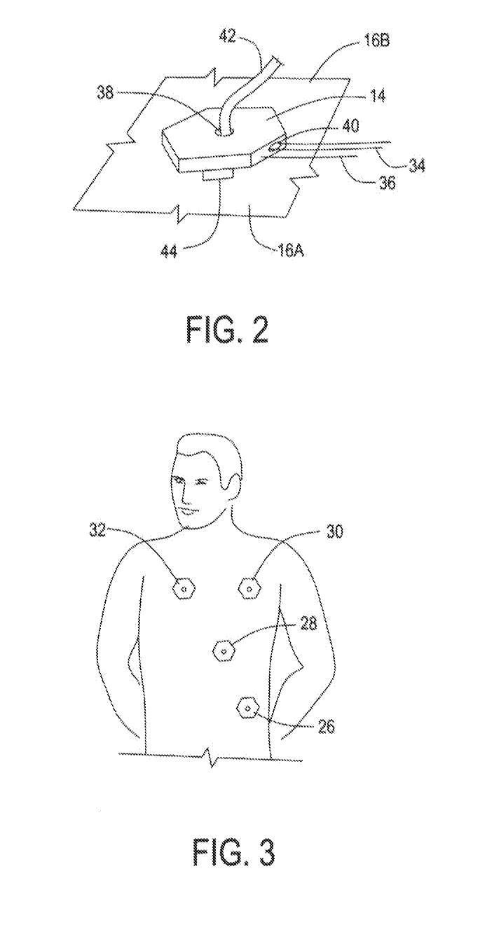

As illustrated in FIG. 2, liner 16 includes an inner liner side 16a and an outer liner side 16b. The inner liner side faces the patient's body, whereas the outer liner side 16b faces externally. In accordance with one embodiment of the invention, bladders 14 are in the shape of hexagons attached together to form a cellular structure as depicted in FIG. 1 and FIG. 2.

Each bladder 14, which in accordance with one embodiment of the invention is formed in a hexagonal shape, includes an air tube outlet 38 that is configured to receive an air tube 42. Air tube 42 is fluidly connected to bladder 14 so that an in-flow of air causes the bladder to expand and an out flow of air causes the bladder to collapse. By controlling the amount of air flowing through air tube 42 the amount of pressure exerted by each bladder on the patient's body can be adjusted. Each bladder 14 also includes outlets for allowing wirings 34 and 36 be extended from inside each bladder to controller 50 as depicted in FIG. 1a.

Each air tube 42 of bladders 14 is coupled to a multi-station miniature valve manifold 60. Valve manifold 60 includes many stations configured to receive corresponding miniature solenoid valves 62. In accordance with one embodiment of the invention, solenoid valves 62 are from the type commercially available from ASCO AM series manufactured by Valworx. Each valve 62 is a two-way closed valve that blocks the air when it is de-energized and allows air to flow in when energized. In accordance with this embodiment of the invention, valve 62 weighs about 1.3 oz and is about 0.75″ in outside diameter and 1.3″ in height. Furthermore, each valve 62 threads to a corresponding station of manifold 60. Manifold 60 includes a common inlet port 66 that allows the air to flow into the manifold. A tube 66 is coupled from controller 50 to manifold 60 allowing air to flow in and out of the manifold's main inlet port.

The two-way switch in each valve 62 is powered externally via controller 50. Control wires for each valve are coupled from the controller to each one of the valves. In accordance with one embodiment of the invention, a separate control wire can be directed from controller 50 to each one of the corresponding valves. In an alternative embodiment, a single control wire can be provided from controller 50, which is then coupled to a multiplexer, such as 68. Each of the valves is then connected to an output port of the multiplexer, so that depending on the number of outputs set to “ON” on the multiplexer, the corresponding valves would be triggered to “open” or “close.”

As such, when a valve 62 is in an open position, air can flow from tube 66 into inlet 60, through valve 62 to a corresponding cellular bladder 14 causing the bladder to inflate. When the valve closes, the air inside the bladder is trapped and keeps the bladder in an inflated position. Again, when the valve is opened, and there is no air flowing through tube 66 in to inlet 60, the air inside the bladder can travel out of the bladder allowing the bladder to deflate.

In accordance with one embodiment of the invention, each bladder 14 is capable of receiving an auscultation microphone such as 44. It is noted that depending on the size of each bladder, an auscultation microphone such as 44 may span the area of more than one bladder 14. Auscultation microphone 44 may be selected from those commercially available, such as 3M Litmann stethoscope. In one embodiment of the invention, the stethoscope is capable of transmitting the sound signals wirelessly to controller 10, such as for example via a Bluetooth communication arrangement.

Stethoscope 44 is capable to operate in both bell and diaphragm mode. As such, when the pressure on a stethoscope 44 is above a threshold value, it operates in the bell mode and when the pressure is below a threshold, it operates in the diaphragm mode. Various sounds for a better diagnosis can be detected when the stethoscope is either in the bell mode or it is in the diaphragm mode.

FIG. 1c illustrates another embodiment of the invention, wherein a single air tube travels through each of the inflatable bladders. In accordance with this embodiment of the invention, instead of the air flowing from mandrel 60 into the bladders in order to increase the pressure within the bladder, the direction of air flow is from within each bladder to the outside. Again the valves are open and closed so as to allow the air to flow out or stay within the bladder. The extendable tube has orifices within each bladder to allow the air flow into the bladder's hollow chamber. When the corresponding valve is closed, the air gets trapped within the bladder forcing it to inflate. Conversely, when the valve is open, the air trapped within the bladder forces out allowing the bladder to deflate. Also shown in FIG. 1c is an optional blood pressure band 120 that may have its own auscultation microphone and air bladders for remotely measuring blood pressure in substantially the same manner as the remote sensor measurements described herein.

FIGS. 1d and 1e illustrate another embodiment of the invention, wherein sensors 142 are physically attached together to form a sensor vest 140 that is separately worn by the patient under a bladder vest. FIG. 1d illustrates the front side of vest 140 as worn by a patient, whereas FIG. 1e illustrates the back side of the vest as worn by the patient. In FIGS. 1d and 1e sensors 142 on vest 140 cover the front with wiring on the back of the patient. However, it is understood that the illustrations in FIGS. 1d and 1e and the description are intended to be exemplary. Sensors 142 may cover the entire vest (and upper body) of the patient, including sensors 142 on the back, sides, under arms, etc. . . . ). See for example alternative FIG. 1f showing sensors 142 covering substantially all of the front and back of vest 140. In fact vest 140 in this and all other embodiments may be extended to lower portions of the patients body including the abdomen and pelvic area.

As illustrated, the front side of the vest is generally divided into two main regions covering the right and left sides of the patient. In each region, a plurality of sensors are disposed in rows of four sensors and columns of 10 sensors. The longitudinal area of the vest separating the two right and left regions is intended to extend along the sternum of the patient. Around the perimeter of the front side a flexible circuit 144 serves as a wiring circuit to connect each of the sensors to a connector 146, which is coupled to controller 50 of FIG. 1c. Flexible circuit 144 also serves as a structural perimeter of the vest.

Shoulder straps 148 and 150 are attached to the top portion of flexible circuit 144 so as to connect the front side of the sensor vest to its back side. A plurality of sensors 140 are also disposed on the back side of the vest, and coupled to flexible circuit 144. Vest 140 includes additional flexible circuits 152, 154 and 156 to couple the sensor connections to connector 146, as well as to provide a more secure fitting for the patient. A belt 158 extends from the lower portion of the front side of the vest to the back side. Shoulder straps 148, side straps 152, 154 and 156, along with belt 158 are securely attach the front side of the vest to its back side after the vest is worn by the patient. It is noted that the structure of vest 140 is commercially available as Heartscape 3D, ECG System, commercially available from Verathon, Inc. (http://www.heartscape.com), the entirety of which is incorporated by reference.

Furthermore, each of the sensors 142, in accordance with one embodiment of the invention, include auscultation sensors, such as those described in reference with FIG. 1a. In accordance with another embodiment of the invention, auscultation sensors such as Hands Free Auscultation commercially available from Universal Biosound, LLC (http://www.universalbiosound.com/), the entirety of which is incorporated by reference.

In accordance with one embodiment of the invention, the mid sternum section of the front side of vest 140 includes a transparent layer 160 having markers such as 162 that connects the two sides of the front side of the vest together. In operation, the person is instructed to make sure that the vest is fitted in such a way that the patient's sternum is aligned along the length of transparent layer 160. To this end, in accordance with one embodiment of the invention, before wearing the vest, the patient is instructed to attach a marker on the area of the skin that covers a specified location of the body, such as on the sternum area and once the vest is worn the patient is further instructed to align the marker on the vest, such as 162 against the marker on the skin to assure the proper positioning of the sensors over the desired locations on the patient's body. It is noted that although this embodiment describes the location of the markers along the patient's sternum, the invention is not limited in this scope and other desired locations may also be marked so as to align the vest when it is worn by the patient.

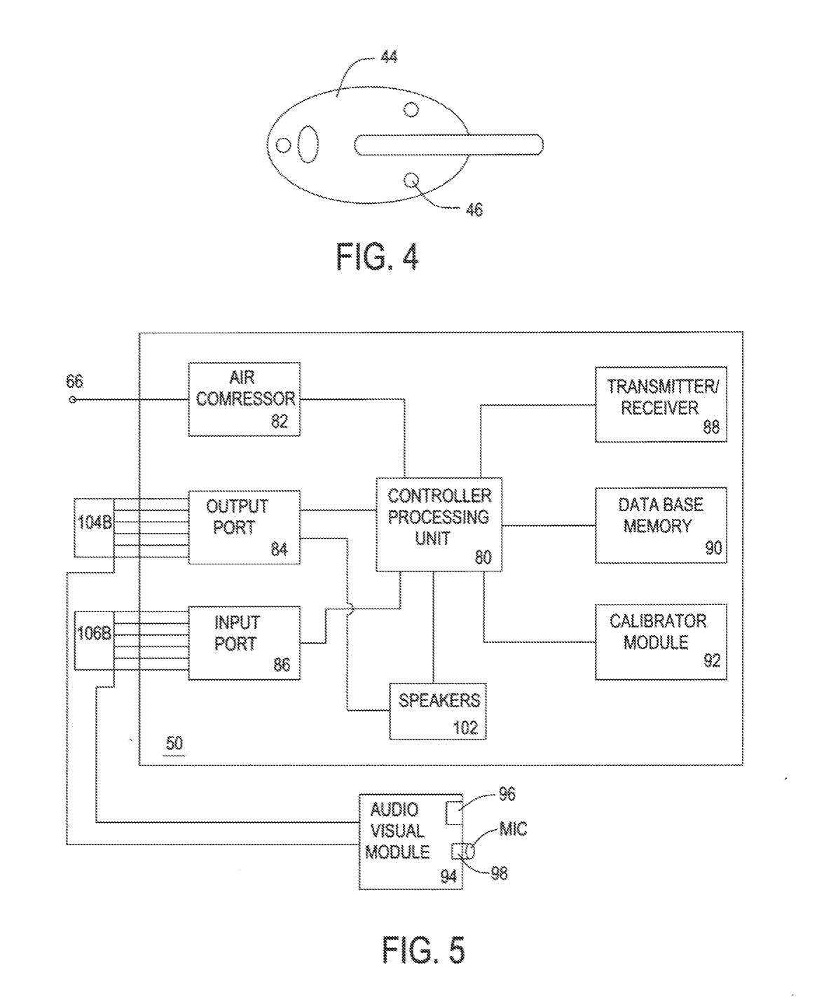

FIG. 4 illustrates a top view of stethoscope 44. As illustrated the stethoscope may have a plurality of fastening clips 46 allowing it to be fastened to a desired portion of vest 10, for example near the locations 26 through 32 depicted in FIG. 3. As with the other embodiments, the locations 26 through 32 are considered exemplary and are not intended to limit the scope of the invention. During the fitting of the vest on a patient, once the vest is worn by the patient, the location of the stethoscopes 44 is checked and adjusted so as to substantially overlap with the correct locations for the diagnosis process such as auscultation.

As explained above, although the embodiments described herein refer mainly to auscultation process, the invention is not limited in scope in that respect. For example, in addition to auscultation, the system is capable of remotely measure the blood pressure of the patient by manipulating the cellular bladders located near the elbow of the patient as illustrated for example in FIG. 1c and described above. Furthermore, instead of auscultation probes, it is possible to fit the vest with other types of sensing probes such as sonogram probes. It is understood that the vest may actually include a mix of both auscultation probes and sonogram probes.

FIG. 5 is a block diagram of controller 50 in accordance with one embodiment of the invention. Controller 50 includes a central processing unit 80 that is programmed to carry out the operation of the controller. An air compressor 82 is coupled to the processing unit 80 so as to provide a controlled air flow to tube 66.

Controller 50 also includes an output port 84 and an input port 86. All control signals necessary to operate the system are provided through output ports 84 and input ports 86. For example, in accordance with one embodiment of the invention, output port 84 provides signals for selectively controlling the opening and closures of solenoids 62. Output port 84 also provides signals for controlling the operation of the sensors, such as stethoscopes 44. For example, in accordance with one embodiment of the invention, control signals to selectively activate or deactivate one or more sensors, such as stethoscopes 44 may be provided via output ports 84.

Similarly, input port 86 of controller 50 is configured to receive signals from various components of vest 10. For example, signals from each sensor 44 are provided to controller 50 via input port 86.

Controller 50 is also coupled to an audio/visual module 94 that includes a camera 96 and a microphone 98. Audio visual module 94 allows the remotely located physician to have a visual of the patient and also enable the patient to communicate with the physician through microphone 98 and speaker 102.

Controller 50 also includes a calibrator module 92 that is configured to control various bladders 14 during the process of fitting a vest for a patient. As various bladders are inflated and deflated, stethoscopes placed on the vest are activated or deactivated by the calibrator module and the sounds within the patient's body are provided through speaker 102. The locations of the stethoscopes are then adjusted until the best fit on the vest provides the optimum sounds.

Controller 50 also includes a database memory 90 coupled to processing unit 80. Database memory 90 is configured to store all the signals generated by the sensors for a desired period of time. This allows the system to also be used not only as a remote monitor, but as a local monitor for later retrieval of the diagnostic signals.

Controller 50 also includes a transceiver 88 for sending and receiving signals to a remote location. In accordance with one embodiment of the invention, transceiver 88 is configured to communicate with a local modem at the patient's location so as to allow communications to the physician's station via the Internet or a direct communication link. Transceiver 88 is also configured to communicate in accordance with Bluetooth and or Wi/Fi protocol to another transceiver 110 located at the patient's premises. For this embodiment, it is transmitter 110 that communicates with the physician's terminal via the Internet or another type of communication channel.

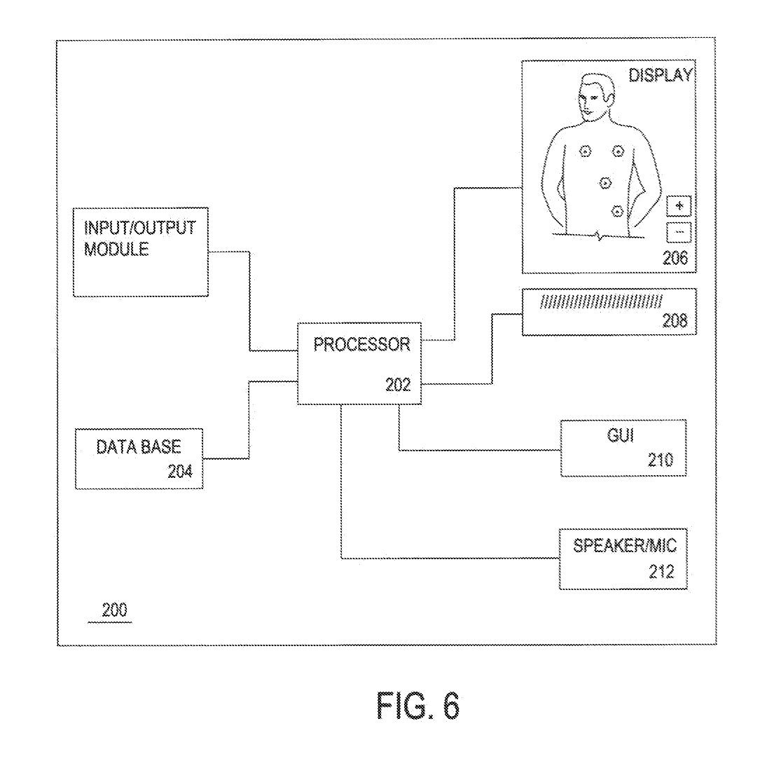

FIG. 6 illustrates a physician station 200 that is configured to allow a health care practitioner remotely receive patient's vital physiological information such as those conducted by an auscultation process. As explained before depending on the type of sensors installed on vest 10 physician station 200 can receive other type of patient physiological information such as sonogram information or echo cardiogram information.

As illustrated in FIG. 6 physician station 200 includes processors 202 configured to control and perform the operations of the station. Database 204 stores all the necessary information regarding each patient monitored or examined by a patient wearing a vest such as vest 10 described above. As such, each patient's information is separately stored in database 202, along with the patient's pertinent information such as the patient's identification information, and auscultation sounds that are recorded during a patient's examination. Database 204 also stores the phonocardiogram of the recorded sounds. A phonocardiogram is a graphical representation of the auscultation sounds that can be helpful in detecting and diagnosing suspected systolic and diastolic murmurs. As such a physician can hear the auscultation sounds live as the patient is being examined, and can also hear them back later by playing back the recorded sounds. The physician can also visually inspect the phonocardiograms for determining the presence of murmurs or other abnormalities.

Physician station 200 also includes display screens 206 and 208, which displays a image of the patient during the examination. This image can be live when camera 96 of audio visual module 94 is interacting with the patient during the examination. As such the physician is enabled to see the patient, and the patient's vest and can communicate with the patient and interact with the patient during the examination.

In accordance with another embodiment of the invention, if no such visual or audio connection exists, a previously stored image of the patient can be retrieved and displayed on screen 206 white the physician station is receiving data from controller 50, or while the physician is retrieving the data from database 204. Meanwhile a different portion of the screen such as 208 displays the sensor signals for the physician's review and analysis.

In accordance with one embodiment of the invention a graphical user interface module 210 allows for the interaction of the physician with the physician station 200 so as to enable the physician to remotely control the operation of the vest.

For example, and in accordance with one embodiment, display 206 is a touch screen that responds to the touching on the screen. For instance by touching on various portions on the image of the body of the patient, the corresponding portions of the vest can be controlled, or the corresponding sensors can be activated. As an example, a particular sensor 44 may be turned on or off using a touch to the sensor. Additionally, by pressing and holding on a touch screen image of a sensor on display 206, the corresponding sensor 44 on vest 10 may be activated, with longer pressing times translating to greater (and/or lesser) pressure behind sensor 44 from bladder(s) 14 to increase (or decrease) signal strength as desired. The operation of touch screen display 206 may control on a sensor by sensor basis or bladder by bladder basis (to potentially cover multiple sensor pressures at the same time).

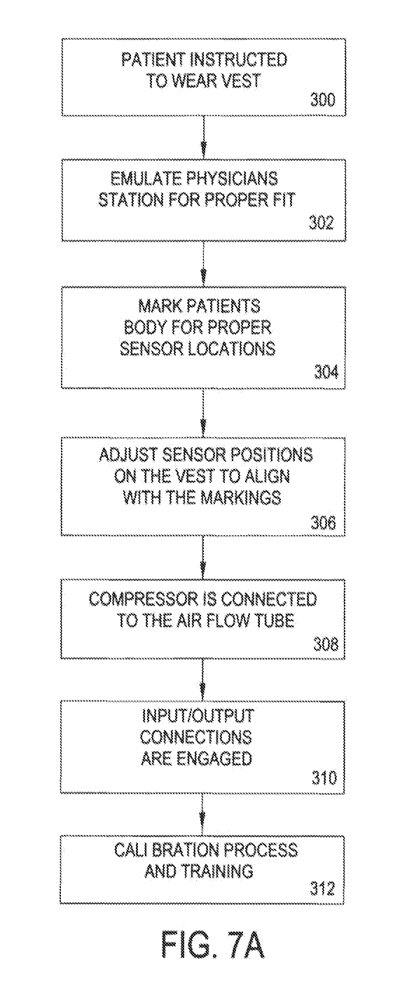

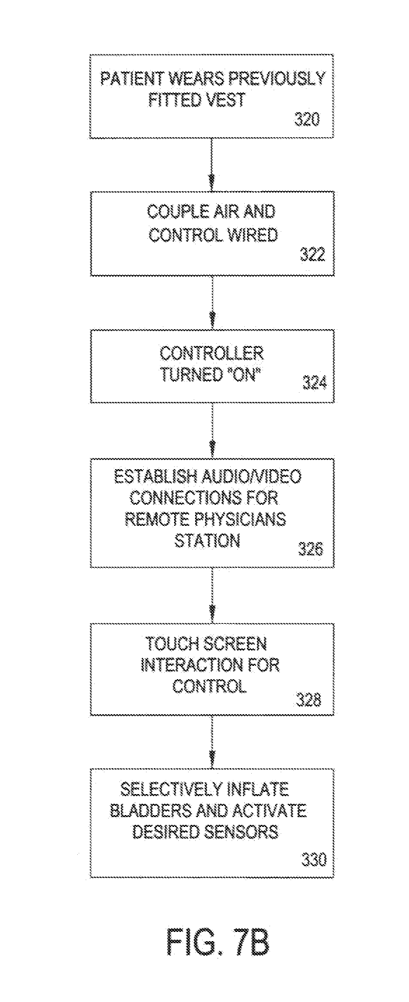

The operation of the system in accordance with one embodiment of the invention in the case of auscultation procedure is explained in more detail hereinafter with reference to FIGS. 7a and 7b. As mentioned before, although the procedure described relates to auscultation, the invention is not limited in scope in that respect and other procedures, such as echo cardiogram, and sonogram are contemplated.

At step 300, the patient wears vest 10 for proper fitting. A healthcare practitioner can visit the patient for fitting the vest. Alternatively, the patient may fit the vest with the aid of the practitioner located remotely at a physician station 200 by communicating via audio visual module 94. In the case that the healthcare practitioner has visited the patient, in accordance with one embodiment, the practitioner at step 302 may be equipped with a portable physician station so as to be able to visualize the signals received from the sensors and emulate the remote station to ensure proper operation.

To this end either the patient or a healthcare practitioner marks the approximate locations on the body to make sure that sensors 44 are disposed as close as possible to the marked locations. For example, the base right location 32, base left location 30, apex 26, and left lateral sternal border 28 on the patient's body are marked at step 304. Sensors 44 have been already placed on the inside surface of vest 10 at approximate locations close to where locations 26, 28, 30 and 32 on the patient's body are predicted to be. The location of sensors 44 are then visually examined to make sure that they are indeed close to the markers. If one or more sensors are not aligned with the markers, the patient or the healthcare assistant can adjust the location of the sensors at step 306. For example, the sensors can be removed and refastened to the inside surface of the vest at a location more closely aligned with the locations 26 through 32 described above.

Once the location of the sensors has been adjusted, at step 308, the patient connects tube 66 from the vest to air compressor unit 82. Similarly connector 104a of vest 10 is coupled to output port 84 via connector 104b, and connector 106b of vest 10 is coupled to input port 86 via connector 106b at step 310. See e.g. FIG. 1c. Next at step 312, processing unit 80 directs calibrator module 92 to test the signals received from the patient. Calibrator module 92 selectively sends signals to control the air flow to cellular bladders 14 so that selected portions of the vest inflate and put pressure on the corresponding sensor. As the pressure is increased, the sensor such as stethoscope 44 begins to pick up the sound signals from the patient. Controller 50 provides the sound signals through speaker 102 for the healthcare practitioner to hear. This process of calibration may include the process of showing the image of the vest (with sensors 44 shown, superimposed over the images of the patient, similar to step 326 described in more detail below to assist in the calibration process.

In accordance with one embodiment of the invention, rather than brining along a portable physician station, it is possible to add the same modules as physician station 200 in controller 50 so as to the healthcare practitioner to visualize the sound signals as explained above in reference with FIG. 6, and control the vest operation via GUI module 210 and a touch screen 206.

Once the healthcare practitioner is satisfied with the operation of vest 10, it is possible to train the patient a few times to wear and take off the vest for proper handling. Also, the patient may be instructed to take the proper postures for sitting, left lateral recumbent position and supine positions. The sounds received in each of these positions are then stored and heard by the practitioner for proper handling and ensuring that the patient is rehearsed sufficiently. Furthermore, at step 312, the blood pressure of the patient can be examined by touching the screen near the arm of the patient so as to activate the cellular bladders in that region. A stethoscope probe in that region is then able to measure the systolic and diastolic pressures by hearing the sounds of the heart beat.

During operation, a patient is contacted at a scheduled appointment time to wear a previously fitted vest 10 at step 320. Air flow tube 66 and control and sensor wires on the vest are then coupled to controller 50 at step 322. Controller 50 is turned on and if the controller includes a video/audio module, it is extended at a position in front of the patient with the vest worn at step 324. At the same time at step 326 physician station 200 establishes an audio/visual communication with the patient. At step 326, the physician station interchangeably or simultaneously displays the live image of the patient, or the previously stored image of the patient with the desired positions of the sensors highlighted on the image. At step 328 physician touches near a desired location that the detection of sounds are intended. At step 330 the corresponding bladders of vest 10 that are proximate to the location of the body corresponding to the areas the physician has touched on the display screen begin to inflate. As the inflation of the bladders continues, at step 332, the physician listens to the corresponding sounds generated by the stethoscope near the desired locations. As mentioned before, all the sounds retrieved from the vest are also stored in a database location for later retrieval, replay and analysis.

While only certain features of the invention have been illustrated and described herein, many modifications, substitutions, changes or equivalents will now occur to those skilled in the art. It is therefore, to be understood that this application is intended to cover all such modifications and changes that fall within the true spirit of the invention.

I claim:

1. An inflatable vest system for remote monitoring and diagnosing a patient's vital health functions comprising:
a wearable vest having an outer surface and an inner surface, said vest being wearable by a patient;
a plurality of bladders having a hollow chamber, said bladders dispersed at selected locations along said vest and disposed adjacent to each other, each of said bladders having an inlet for selectively receiving and releasing air within its chamber;
a plurality of adjustable sensors attached to said vest and disposed within the vicinity of desired locations on the patient's body, said sensors exerting pressure on said desired locations as a function of the air pressure of at least one bladder most closely located to said sensor;
said vest receiving control signals from a remote physician system, said control signals selectively activating and deactivating desired one of said sensors, said control signals further selectively increase and decrease air pressures in desired one of said bladders, such that said vest provides desired patient information to said remote physician station.

2. The vest in accordance with claim 1, further comprising an electrically activated valve for allowing air to flow in and out of said bladders.

3. The vest in accordance with claim 2, further comprising an air tube extending from an inlet port of a cellular bladder to a valve located on a manifold that is configured to receive a plurality of said valves.

4. The vest in accordance with claim 3 wherein said manifold has a main inlet tube from receiving air, such that for each valve that is activated to be open, air flows through said valve through said tube to a corresponding bladder.

5. The vest in accordance with claim 1 further comprising an air inlet interconnect coupled to an air compressor for receiving air from said compressor so as direct air flow to said bladders.

6. The vest in accordance with claim 5, further comprising a connector for connecting valve control wires and sensor wires to a controller unit configured to control the operation of said valves and to receive said sensor signals.

7. The vest in accordance with claim 6 wherein said sensors are selected from the group consisting of electronic stethoscopes, auscultation heads and sonography probes.

8. The vest in accordance with claim 7 wherein the sounds detected by said stethoscopes depend on the pressure exerted on said stethoscopes against the patient's body.

9. The vest in accordance with claim 8 wherein said stethoscopes are located substantially over said patient's Base Right, Base Left, Apex and Left Lateral Stermal Border.

10. The vest in accordance with claim 9 wherein signals detected by said stethoscopes are transmitted to said physician station.

11. The vest in accordance with claim 10, wherein the operation of said vest is controlled at said physician station where an image corresponding to said patient is displayed on a touch screen.

12. The vest in accordance with claim 11, wherein one or more of said bladders are inflated as a corresponding location of said touch screen is pressed.

13. The vest in accordance with claim 12, wherein the sound detected by a stethoscope is replayed at said physician station for monitoring and diagnosis.

14. The vest in accordance with claim 13 where all information gathered by said stethoscopes are stored in a database coupled to said physician station.

15. The vest in accordance with claim 14 wherein said physician station displays sounds detected by said stethoscopes as phonocardiograms.

16. A method for remote monitoring and diagnosing a patient's vital health functions using an inflatable vest system, said method comprising the steps of:
supplying a wearable vest to a patient, said wearable vest having an outer surface and an inner surface, said wearable vest further having a plurality of bladders with hollow chambers, said bladders dispersed at selected locations along said vest and disposed adjacent to each other, each of said bladders having an inlet for selectively receiving and releasing air within its chamber;
exerting pressure by sensors on desired locations as a function of the air pressure of at least one bladder most closely located to said sensor, where said sensors include a plurality of adjustable sensors attached to said vest and disposed within the vicinity of said desired locations on the patient's body;
sending control signals to said vest from a remote physician system, said control signals selectively activating and deactivating desired one of said sensors, said control signals further selectively increase and decrease air pressures in desired one of said bladders, such that said vest provides desired patient information to said remote physician station.
